(19) United States
(12) Patent Application Publication
(10) Pub. No.: US 2007/0267322 A1
(43) Pub. Date: Nov. 22, 2007

(54) Packaged absorbent product having window on package

(76) Inventors: Kazushige Kishida, Kobe (JP); Chisako Tsunawaki, Nishinomiya (JP); Mami Kobayashi, Nishinomiya (JP)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/796,508

(22) Filed: Apr. 27, 2007

Related U.S. Application Data
(60) Provisional application No. 60/800,618, filed on May 16, 2006.

(51) Int. Cl. B65D 25/54; B65D 73/00; A61B 19/02

(52) U.S. Cl. 206/776; 206/440; 206/494; 206/457

(57) ABSTRACT

A packaged absorbent product including a package having at least one window and a plurality of absorbent articles contained in the package. At least one of the absorbent articles has a first graphic printed on a part of the article. The window has a background area and a second graphic. The first and second graphics have a common visual configuration.

### CROSS REFERENCE TO RELATED APPLICATION

This application claims the benefit of U.S. Provisional Application No. 60/800,618 filed on May 16, 2006.

### FIELD OF THE INVENTION

The present invention relates to packaged absorbent products. More particularly, the present invention relates to a packaged absorbent product including a package for absorbent articles which has a window.

### BACKGROUND OF THE INVENTION

Disposable absorbent articles such as sanitary napkins, pantiliners, baby diapers, and incontinent diapers are pads or devices that are typically worn in the crotch region of an undergarment. More specifically, sanitary napkins and pantiliners, for example, are worn by women in a pair of panties and are normally positioned between the wearer's legs, adjacent to the perineum area. These are designed to absorb and retain body fluids or discharges (e.g., menses and urine) from the body of women and to reduce or prevent soiling of the body and/or clothing. Often, such absorbent articles are contained in a package for shipping and sale.

Recent developments for disposable absorbent articles have focused on not only improvement of their performance functions (e.g., superior absorbency, leakage protection and comfort) but their aesthetic features. For example, absorbent articles which have an aesthetic feature are disclosed in WO 2004/006818 published on Jan. 24, 2004. This publication discloses an absorbent article having a graphic printed at the backsheet which is said to provide an emotional benefit to users by decreasing users' melancholic mood.

The aesthetic design of disposable absorbent articles may be important since it tends to affect consumer's impression about the articles in the market. In conventional disposable absorbent articles (typically sanitary napkins and pantiliners), each pad may be wrapped individually by a wrapper sheet. Since these sheet materials normally have high opacity, consumers can not clearly see a graphic, if any, printed on the article through the wrapper sheet. This issue becomes more relevant when absorbent articles are contained in a package (e.g., a plastic bag) for shipment and sale. Such a package is typically formed by a package member which also has high opacity (or even no transparency), and thus it is very difficult to show consumers the graphic printed on a portion of the article through the package member and the wrapper sheet.

Thus, it would be desirable for a package for absorbent articles that can provide an image showing at least a part of the contents of the package.

It would also be desirable to provide a package for absorbent articles that has a window and that can show a graphic on at least one of the articles in the package.

### SUMMARY OF THE INVENTION

In one aspect, the invention is directed to a packaged absorbent product including a package having at least one window and a plurality of absorbent articles contained in the package. At least one absorbent article has a first graphic on a part of the article. The window has a background area and a second graphic. The first and second graphics have a common visual configuration.

In another aspect, the invention is directed to a packaged absorbent product including a package having at least one window and a plurality of absorbent articles contained in the package. At least one of the absorbent articles includes a backsheet having a graphic thereon. The window has a transparency or translucency so that the graphic of at least one absorbent article can be seen through the window.

The foregoing answers the need for a package for absorbent articles that can provide an image showing at least a part of the contents of the package.

The foregoing may also answer the need for a package for absorbent articles that has a window and that can show a graphic on at least one of the articles in the package.

These and other features, aspects, and advantages of the present invention will become evident to those skilled in the art from reading of the present disclosure.

### BRIEF DESCRIPTION OF THE DRAWINGS

FIG. 1 is a simplified perspective illustration of a packaged absorbent product, which is one embodiment of the present invention.

### DETAILED DESCRIPTION OF THE INVENTION

Herein, “comprise” and “include” mean that other elements and/or other steps which do not affect the end result can be added. Each of these terms encompasses the terms “consisting of” and “consisting essentially of”.

Herein, “absorbent article” refers to articles which absorb and contain body exudates or discharges such as body fluids, and is intended to include sanitary napkins, pantiliners, tampons, interlabial devices, diapers (both for baby and adult incontinent), and adult incontinent pads (and other articles worn in the crotch region of a garment).

Herein, “disposable” refers to articles which are intended to be discarded after a single use, composted, or otherwise disposed of in an environmentally compatible manner. (That is, they are not intended to be laundered or otherwise restored or reused as an absorbent article.) Herein, “sanitary napkin” refers to articles which are worn by females adjacent to the pudendal region which are intended to absorb and contain the various exudates which are discharged from the body (e.g., blood, menses, and urine).

Herein, “package” refers to a means for containing one or more an absorbent articles. Packages are formed from a package member. The package can take any size, shape and structure known in the art. In certain embodiments, the package can be a flexible bag having a cube like shape which contains a plurality of absorbent articles. Alternatively or additionally, the packages can include a box having a cubic shape or a columnar shape.

Herein, “window” refers to portions or partial areas of packages that can provide an image showing at least a part (e.g., the outline, the thickness, or a graphic(s)) of the contents of the package. In certain embodiments, the image can be provided directly from the contents of the package through the window, i.e., a part of the contents can be directly seen through the window. Alternatively or additionally, the image can be provided indirectly, i.e., it can be provided by the printed graphic which provides an image showing at least a part (e.g., the outline, the thickness, or a graphic(s)) of the contents of the package.

In one embodiment, the image can be provided both directly and indirectly within the window, i.e., one part of the image can be directly seen through the window, while the other part of the image can be provided by the printed graphic in the window.

In one embodiment, the window can be formed in at least the front view area of the package (e.g., the front panel for a cubic like shaped package). The window can have no transparency, some or complete transparency or translucency, or a combination thereof which changes depending on the areas. When the window has no transparency, the graphic disposed in the window may provide the image showing (i.e., indicating) at least a part of the contents of the package. Alternatively, when the window has some or complete transparency or translucency, a part of the contents of the package can be directly seen through the window.

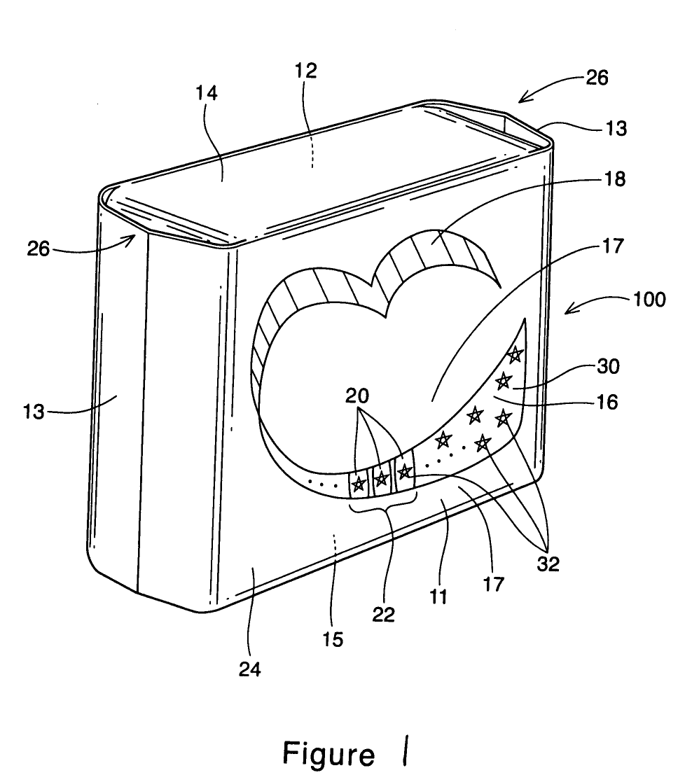

The window can be formed by any means known in the art. In one embodiment, the window can be formed by differentiating the degree of the transparency or translucency at the window area from the surrounding area which has higher opacity or no transparency. In the embodiment shown in FIG. 1, for example, the window area is indicated by the reference number 16, while the surrounding area is indicated by the reference number 17. Such a differentiation can be made by using different inks or different amount of ink(s) between the window area and the surrounding area. In other embodiments, the window can be formed by applying no paint in the window area so that it has high transparency, while applying a paint which gives the surrounding area translucency, higher opacity or no transparency. Alternatively, the window having no transparency can be formed by making a color difference by printing inks between the window area and the surrounding area. Or, the window can be formed by printing a line by an ink(s) between the window area and the surrounding area. The window can also be formed by providing different materials on the package, e.g. transparent or translucent materials for the window area 16 and other materials for the surrounding area 17.

Herein, “graphic” refers to patterns that are constituted by a figure(s), i.e., a line(s), a symbol(s) or a character(s), a color difference(s) or transition(s) of two (or more) colors, and/or the like. In certain embodiments, the graphic has an aesthetic design and/or color(s) that can provide emotional benefit(s) when the absorbent article having the graphic is seen or viewed by users. Examples of such aesthetic designs include, but not limited to, plants such as flowers (e.g., a dandelion), animals, cartoon characters, seasonal things or goods such as snowmen, landscapes, and the like. In addition or alternatively, the graphic can include other element(s), for example, an information tip(s) such as the size, the type (e.g., wings/no-wings and the day/night use) for sanitary napkins, the boy/girl use for baby diapers, and the brand name or its logo of the absorbent article, and the like. The graphic can be provided by print or any other known means for providing a graphic. If printing is used, the printing can be any conventional printing method or technology known in the art, including, but not limited to, gravure printing, flexo printing, offset printing, ink jet printing, and the like.

Each graphic has a visual configuration. The visual configuration, as used herein, does not necessarily include the size of the graphic. Thus, for example, two graphics may be considered to have a common visual configuration if they are identical or if they are only different in their sizes.

Herein, “front view area” refers to primary surface areas of packages which are intended to face consumers when in the market displayed for sale. In many embodiments, the front view area has the largest indication of a brand name or a product name on the outer surface of the package.

The absorbent article or product may take any form. In certain embodiments as noted above, the absorbent article may be in the form of a feminine hygiene article or diaper. Certain exemplary diapers and feminine hygiene articles include a topsheet, a backsheet and an absorbent core. Other articles, such as, for example, tampons, may include absorbent cores, overwraps, withdrawal cords and/or other materials. In any case, the topsheet, backsheet, absorbent core or other materials can be formed from any suitable materials known in the art.

The topsheet can be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the body of the wearer. A suitable topsheet can be made of various materials such as woven and nonwoven materials; apertured film materials including apertured formed thermoplastic films, apertured plastic films, and fiberentangled apertured films; hydro-formed thermoplastic films; porous foams; reticulated foams; reticulated thermoplastic films; thermoplastic scrims; or combinations thereof.

The backsheet generally prevents external leakage of exudates absorbed and contained in the absorbent articles. Flexible materials suitable for use as the backsheet include, but are not limited to, woven and nonwoven materials, laminated tissue, polymeric films such as thermoplastic films of polyethylene and/or polypropylene, composite materials such as a film-coated nonwoven material, or combinations thereof. In certain embodiments, the backsheet may have a thickness of from about 0.012 mm (0.5 mil) to about 0.081 mm (3.4 mils).

The absorbent core is typically positioned between the topsheet and the backsheet. Herein, “absorbent core” refers to a material or combination of materials suitable for absorbing, distributing, and storing aqueous fluids such as urine, blood, menses, and water found in body exudates. The size and shape of the absorbent core can be altered to meet absorbent capacity requirements, and to provide comfort to the wearer/user. The absorbent core suitable for use in the present invention can be any liquid-absorbent material known in the art for use in absorbent articles, provided that the liquid-absorbent material can be configured or constructed to meet absorbent capacity requirements.

Non-limiting examples of liquid-absorbent materials suitable for use as the absorbent core include comminuted wood pulp which is generally referred to as airfelt; creped cellulose wadding; absorbent gelling materials including superabsorbent polymers such as hydrogel-forming polymeric gelling agents; chemically stiffened, modified, or crosslinked cellulose fibers; meltblown fibers; synthetic fibers including crimped polyester fibers; tissue including tissue wraps and tissue laminates; capillary channel fibers; absorbent foams; absorbent sponges; synthetic staple fibers; peat moss; or any equivalent material; or combinations thereof.

The topsheet, the backsheet, and the absorbent core can be assembled in a variety of configurations, sizes and shapes known in the art. Examples of configurations are described in U.S. Pat. Nos. 4,950,264 and 5,009,653 issued to Osborn; U.S. Pat. No. 4,425,130 issued to DesMarais; U.S. Pat. Nos. 4,589,876 and 4,687,478 issued to Van Tilburg; and U.S. Pat. No. 5,234,422 issued to Sneller, et al.

In certain embodiments, each of the absorbent articles 20 can be individually packaged thereby forming an individual wrapper structure or a pouch structure. Specifically, in the case where the absorbent articles are sanitary napkins, pantiliners, interlabial devices or tampons, each article wrapped individually by a wrapper sheet or contained in a pouch. The wrapper sheet or pouch can be formed by a package member which is described in more detail hereinafter. One example of an individual wrapper structure for a sanitary napkin having flaps is disclosed in U.S. Pat. No. 6,074,376 issued to Mills on Jun. 13, 2000.

Alternatively, the absorbent articles can be packaged in a wrapper with one or more other articles or provided with no wrapper or pouch The absorbent articles can be stored in the package in any suitable configuration. For example, when the absorbent articles are sanitary napkins, each sanitary napkin can be folded along one or more folding lines. Examples of manners or structures for folding and wrapping feminine hygiene articles (e.g., sanitary napkins) are disclosed in U.S. Pat. No. 6,074,376 issued to Mills on Jun. 13, 2000 and U.S. Pat. No. 5,569,228 issued to Byrd et al. on Oct. 29, 1996.

At least one of absorbent articles has a graphic (i.e., a first graphic) disposed on a part of the article. In certain embodiments, each absorbent article has (i.e., all the absorbent articles in the package have) such a graphic disposed on a part of the article. The graphic can be disposed on any part (or the whole area) of any component member(s) of the absorbent article. For example, there can be graphics disposed on the topsheet, backsheet, absorbent core, the wrapper sheet or the pouch (if it is employed), a panty fastener adhesive disposed on the backsheet, a cover sheet (e.g., a release paper) or any other component of the article. The graphic can be located at either the body facing surface or the garment facing surface of the component member(s) of the absorbent article. While the graphic can be disposed on only a small part of a component member in the absorbent article, it can be disposed on the whole area of a component member or it can be a repeating graphic element. For example, in the embodiment shown in FIG. 1, each absorbent article has a graphic printed of the body facing surface (or the garment facing surface if desired) of the backsheet. In alterative embodiments, each absorbent article has a graphic on either the wrapper sheet or the cover sheet for the panty fastener adhesive.

FIG. 1 is a simplified perspective illustration of a packaged absorbent product, i.e., a package 100 containing absorbent articles 20, which is one embodiment of the present invention. In this embodiment, the package 100 has a cube like shape and contains a plurality of disposable absorbent articles 20. In certain embodiments, the absorbent articles 20 may be stacked to form at least one stack 22 in the package 100. However, in certain embodiments, the absorbent articles 20 are not stacked but simply placed in the package. The package 100 shown in FIG. 1 is a flexible bag which can be formed from a continuous package member. The side portions of the package 100 can be closed by forming gusset structures 26 at the side panels 13.

The package 100 includes a front panel 11, a rear panel 12 opposed to the front panel 11, side panels 13 which connects the front and rear panels 11 and 12, a top panel 14 which connects the front, rear, and side panels 11, 12 and 13, and a bottom panel 15 opposed to the top panel 14. Each pair of the front and rear panels 11 and 12, the side panels 13 and the top and bottom panels 14 and 15 can be substantially planar as shown in FIG. 1. These panels 11, 12, 13, 14 and 15 have an outer surface 24 and an inside surface. In this embodiment, the front panel 11 corresponds to the front view area and has the largest indication of a brand name or a product name (not shown) within the outer surface 24 of the package 100.

The package 100 of the present invention further includes a window(s) 16. The window 16 can be provided on any of the panels 11, 12, 13, 14 and/or 15 of the package 100. In one embodiment, the window 16 can be provided on at least the front panel 11. If desired, more than one window 16 can be provided in the front and/or the other panel(s) of the package 100. For example, an additional window(s) may be provided in the front panel 11, side panel(s) 13 and/or the rear panel 12. If desired, the window 16 can be extended from one panel to another adjacent panel(s), e.g., from the front panel 11 to the side panel(s) 13.

The window 16 of the present invention has a background area 30 and a second graphic 32. The first and second graphics have a common visual configuration.

The second graphic 32 can be located on either the outer surface 24 or the inner surface of the package 100. The second graphic 32 can be disposed on a discrete part of the window 16, across the whole area of the window 16 or by repeating a graphic element.

The window 16 can take any shape such as a circle, a square, a rectangle, a trapezoid, an ellipse, a triangle or any other shape. An irregular shape is shown in FIG. 1. The window 16 can have any sizes or dimensions. In certain embodiments, the window 16 is configured such that at least some of the contained absorbent articles 20 can be seen through the window 16.

The window 16 can show consumers the design of the contained disposable absorbent articles 20 in the package 100. This is beneficial at the point of purchase in particular when a graphic is disposed on a part of the absorbent articles 20 such that consumers can recognize it through the window 16.

In the embodiment shown in FIG. 1, the window 16 can be formed by applying no paint, ink or other color in the area of the window 16 so that it has a very high transparency, while applying a paint, ink or other color which makes the surrounding area of the window 16 less transparent. The rest of the front panel 11 can have one or more colors, lines, figures, ornamental designs, pictures, symbols, texts (e.g., a brand name and/or product name), and/or any combination thereof. (In the embodiment shown in FIG. 1, for example, a graphic which is a part of the front panel 11 outside the window 16 is indicated by the reference number 18.) In one embodiment, at least a portion of the window 16 has little or no transparency. In certain embodiments, the whole area of the window 16, i.e., both the background area 30 and the second graphic 32 have little or no transparency. Since no part of the absorbent article 20 can be actually seen through the window 16, the absorbent articles 20 contained in the package 100 can be protected from a UV light. However, since the first and second graphics have a common visual configuration, consumers can recognize what graphic or graphics are on the absorbent articles 20 in the package 100 by looking at the second graphic 32 printed on the window 16.

Additionally, the background area 30 and the second graphic 32 can have similar colors to (or substantially same as) those of the backsheet (or other element of the absorbent article) and/or the first graphic on the absorbent articles 20, respectively. Since the window 16 of this embodiment can show the second graphic 32 which may be the same (including the color combination) as the first graphic on the backsheet, etc., the consumers can get a more realistic image of the graphic on the absorbent articles 20.

In one embodiment, the window 16 has certain translucency (i.e., the window is translucent) so that a part of the absorbent article 20 can be seen through the window 16. Herein, a “translucent window” refers to a window which is formed on one panel of the package and can show the outline or the thickness of the absorbent articles contained in the package therethrough but does not clearly show the graphic. The translucent window can have a color but it should have enough translucency so that the outline of the absorbent articles 20 contained in the package can be seen through the window 16. Although the first graphic on the absorbent articles 20 can not be clearly seen through the window 16 due to its translucency, this embodiment may be desired since the window 16 of this embodiment can actually show the outline of the absorbent articles, the consumers can get a better image of the absorbent articles 20 with the graphic. It should be noted that the translucent window can protect the absorbent articles 20 contained in the package 100 from a UV light.

In another embodiment, the window 16 may be a transparent window which shows at least a part of the absorbent articles 20 contained in the package 100 through the window 16. Herein, “transparent” refers to area(s) or portion(s) which are formed on at least one panel of the package that can clearly show at least a part of the outline, the thickness and/or the printed graphic of the absorbent articles 20 contained in the package 100. If desired, the transparent window can have color. In certain embodiments, the transparent window can have a graphic, the second graphic, which can have a common visual configuration with the first graphic. For example, the first and second graphics can have a common visual configuration, e.g., a common dandelion flower. In certain embodiments, the first and second graphics can jointly form a combined image or picture which is preferred by consumers. For example, the first and second graphics may have a different visual configuration e.g., a yellow dandelion flower and its green leaves, respectively. Such first and second graphics can be any graphic(s), including but not limited to plants or flowers, animals, cartoon characters, etc. It should be noted that the transparent window can protect the absorbent articles 20 contained in the package 100 from a UV light.

In certain embodiments, each absorbent article 20 includes a component having a graphic disposed thereon, and the transparency or translucency of the window can be configured such that the graphic of at least one absorbent article can be seen through the window 16.

In certain embodiments, one or more of the absorbent articles can include a wrapper sheet (i.e., a package member) which wraps the respective absorbent article 20. In such embodiments, the total transparency or translucency of the window and wrapper sheet can be controlled such that the graphic of the absorbent article can be seen through the wrapper sheet and the window.

Further, one or more of the absorbent articles can include a panty fastener adhesive disposed on the backsheet and a cover sheet (e.g., a release paper) for protecting the adhesive. In such embodiments, the total transparency or translucency of the panty fastener adhesive, the cover sheet, the wrapper sheet, and the window can be controlled such that the graphic of the absorbent article can be seen through the panty fastener adhesive, the cover sheet, the wrapper sheet, and the window.

Package members for the aforementioned individual wrapper, the pouch and the package may include a thin plastic film material, a nonwoven material and/or a paper material. Some exemplary suitable thermoplastic polymers that may be used in films or nonwoven materials include: polyolefin such as polyethylene (PE), including a liner low density polyethylene (LLDPE), a low density polyethylene (LDPE), a ultra low density polyethylene (ULDPE), a high density polyethylene (HDPE), a polypropylene, and a mixture thereof. Other suitable thermoplastic polymers which may also be used include, but are not limited to a polyester, a polyurethane, a compostable or biodegradable polymer, a thermoplastic elastomer, and a metallocene catalyst-based polymer. In certain embodiments, materials for the package member can be liquid and moisture impermeable so that the package can protect the absorbent articles from being affected by the moisture outside of the package 100. The materials may be non-biodegradable, non-recyclable, biodegradable, or recyclable.

It should be noted that a different material(s) can be used at the window area if a chosen material per se can not provide an expected optical property (e.g., transparency or translucency) at the window area. For example, when a cardboard material which has no transparency is chosen as a package member, a thermoplastic polymer material (e.g., polyethylene) having a transparency or translucency can be used at the window area.

The disposable absorbent articles 20 contained in the package 100 can include the same or two (or more) different types or designs of absorbent articles 20. In certain embodiments, the disposable absorbent articles 20 contained in the package 100 are feminine hygiene articles such as sanitary napkins and pantiliners. In the embodiment shown in FIG. 1, a single type (or one design) of sanitary napkins is contained in the package 100.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A packaged absorbent product, comprising:
a package having at least one window; and
a plurality of absorbent articles contained in the package;
at least one absorbent article has a first graphic disposed on a part of the article;
the window having a background area and a second graphic;
wherein the first and second graphics have a common visual configuration.

2. The packaged absorbent product of claim 1, wherein each absorbent article is wrapped by a wrapper sheet or contained in a pouch, and wherein the first graphic cannot be seen from the outside of the package.

3. The packaged absorbent product of claim 2, wherein each absorbent article includes a backsheet having the first graphic printed, and wherein the background area includes a color which is similar to that of the backsheet.

4. The packaged absorbent product of claim 1, wherein the window is not transparent.

5. The packaged absorbent product of claim 1, wherein the window is transparent or translucent so that at least a part of the absorbent article can be seen through the window.

6. The packaged absorbent product of claim 1, wherein the absorbent article is a sanitary napkin, a pantiliner, a tampon, an interlabial device, a diaper, or an adult incontinent pad.

7. A packaged absorbent product, comprising:
a package having at least one window; and
a plurality of absorbent articles contained in the package, at least one absorbent article including a backsheet having a first graphic disposed thereon;
wherein the transparency or translucency of the window is controlled such that the first graphic of the at least one absorbent article can be seen through the window.

8. The packaged absorbent product of claim 7, wherein each absorbent article further includes a wrapper sheet which wraps the respective absorbent article, and the total transparency or translucency of the window and wrapper sheet is controlled such that the first graphic of the absorbent article can be seen through the wrapper sheet and the window.

9. The packaged absorbent product of claim 8, wherein each absorbent article further includes a panty fastener adhesive disposed on the backsheet and a cover sheet for protecting the adhesive, and wherein the total transparency or translucency of the panty fastener adhesive, the cover sheet, the wrapper sheet, and the window are controlled such that the first graphic of the absorbent article can be seen through the panty fastener adhesive, the cover sheet, the wrapper sheet, and the window.

10. The packaged absorbent product of claim 7, wherein the window has a second graphic which can be combined with the first graphic of the absorbent articles so that the two graphics can jointly form a combined image or picture.

11. The packaged absorbent product of claim 7, wherein the absorbent article is a sanitary napkin, a pantiliner, a tampon, an interlabial device, a diaper, or an adult incontinent pad.
